(19) United States
(12) Patent Application Publication
(10) Pub. No.: US 2026/0266795 A1
(43) Pub. Date: Sep. 10, 2026

(54) METHOD AND SYSTEM FOR PREDICTING SALINITY IN TIDAL REACHES BASED ON RANDOM FOREST AND EMPIRICAL MODE DECOMPOSITION

(71) Applicant: SUN YAT-SEN UNIVERSITY, Guangzhou (CN)

(72) Inventors: Hanliang HUANG, Guangzhou (CN); Jingwen ZHANG, Guangzhou (CN); Jingru JI, Guangzhou (CN); Zheng KANG, Guangzhou (CN); Kairong LIN, Guangzhou (CN)

(21) Appl. No.: 19/455,323

(22) Filed: Jan. 21, 2026

(30) Foreign Application Priority Data
Mar. 6, 2025 (CN) 2025102619724

(51) Int. Cl. G01N 33/18; G06F 17/10

(52) U.S. Cl. CPC G01N 33/18; G06F 17/10

(57) ABSTRACT

The present disclosure relates to the technical field of salinity prediction and management of tidal reaches and provide a method and system for predicting and managing salinity of tidal reaches based on random forest and empirical mode decomposition. The method includes: acquiring dry season data; acquiring mutual information corresponding to different lag times according to the dry season data; establishing a tidal reach salinity prediction model according to the dry season data and the mutual information corresponding to the different lag times; and obtaining tidal reach salinity prediction information according to the tidal reach salinity prediction model, such that the tidal reach salinity prediction information can be used for decision-making in dispatching upstream reservoirs to increase discharge, closing intake gates and activating backup water sources, and optimizing industrial and domestic water-use scheduling along the riverbanks to ensure water supply safety.

### CROSS-REFERENCE TO RELATED APPLICATION

This application is based on and claims the benefit of priority from Chinese Patent Application No. 202510261972.4, filed on 6 Mar. 2025, the entirety of which is incorporated by reference herein.

### TECHNICAL FIELD

The present disclosure relates to the technical field of salinity prediction of tidal reaches for managing urban water resources, in particular to a method and system for predicting salinity in tidal reaches based on random forest and empirical mode decomposition.

### BACKGROUND

Currently, in regions of tidal reaches where saltwater intrusion occurs frequently, seawater backflow raises the salinity at water intake points of municipal water plants, causing serious impacts on domestic water use and urban water supply safety. Especially during the dry season, when upstream runoff decreases, saltwater intrusion often results in tap water in some coastal cities failing to meet standards. For example, in the Pearl River Delta region, water intake has repeatedly been suspended due to saltwater intrusion, leading to difficulties in urban water supply scheduling and affecting residents' daily lives.

Therefore, accurately predicting salinity variation trends in tidal reaches can help water resources management authorities take early countermeasures, such as regulating reservoir discharge, controlling the opening and closing of barrage sluices, or modifying water supply scheduling, thereby mitigating the adverse impacts of saltwater intrusion on people's livelihood and the ecological environment.

In the conventional technology, it is common to use the following dedicated devices or instruments to collect required data, for example but not limited to: 1) float-type tide gauges; 2) photoelectric anemometers; 3) vessel-mounted Acoustic Doppler Current Profilers (ADCPs) or sonar systems; 4) Conductivity-Temperature-Depth (CTD) instruments. The above dedicated devices may refer to, but are not limited to, for example, GB189706567A, U.S. Pat. Nos. 4,940,330A, 7,420,875B1, and 7,259,566B2.

In the conventional technology, salinity in estuaries is generally predicted using a single optimal model, which, however, has significant shortcomings when dealing with non-stationary data. Traditional models, such as hydrodynamic models and machine learning models, cannot effectively cope with complex, nonlinear, and non-stationary time series data. Generally, such methods cannot well separate different frequency components (such as periodicity and noise) in a signal, resulting in low prediction accuracy and poor stability, thus impacting water resource management actions.

Therefore, an improved salinity prediction method and system for tidal river reaches is required to rapidly provide more accurate salinity forecasts for improved management of water resources due to tidal river reaches. The more accurate salinity predictions can be directly applied to urban water-supply and estuarine-regulation systems so that the systems can issue early warning signals and water authorities can promptly take a series of countermeasures against salt tides (for example, measures referred to in WO2004080793A1 and WO2020160655A1). This makes urban water-supply and estuarine-regulation systems more reliable and provides greater assurance for residents' domestic water supply.

### SUMMARY

A main objective of embodiments of the present disclosure is to provide a method and system for predicting salinity of tidal reaches based on random forest and empirical mode decomposition.

The following solutions are employed in the present disclosure.

In accordance with one aspect of the present disclosure, an embodiment provides a method for predicting salinity in tidal reaches based on random forest and empirical mode decomposition, including:
acquiring dry season data;
acquiring mutual information corresponding to different lag times according to the dry season data;
establishing a tidal reach salinity prediction model according to the dry season data and the mutual information corresponding to the different lag times; and
obtaining tidal reach salinity prediction information according to the tidal reach salinity prediction model.

The dry-season data may be collected by dedicated devices or instruments at the monitoring stations, for example but not limited to: 1) float-type tide gauges; 2) photoelectric anemometers; 3) vessel-mounted Acoustic Doppler Current Profilers (ADCPs) or sonar systems; 4) Conductivity-Temperature-Depth (CTD) instruments.

Further, the step of acquiring dry season data includes:
collecting historical data of a target station, where the historical data includes historical tidal level factor data, historical wind speed data, historical upstream runoff data, and historical salinity data;
the historical tidal level factor data includes a daily average tidal level, a daily maximum tidal level, a daily minimum tidal level, and a tidal range;
the historical wind speed data includes an average wind speed, a maximum wind speed, and an extreme wind speed; and
sorting the historical data into a daily scale and sifting the historical data to obtain the dry season data, where the dry season data includes dry-season tidal level factor data, dry-season wind speed data, dry-season upstream runoff data, and dry-season salinity data.

Further, the step of acquiring mutual information corresponding to different lag times according to the dry season data includes:
obtaining an input variable and an output variable according to the dry season data, where the input variable includes tidal level, wind speed, runoff, and the output variable includes salinity; and
obtaining the mutual information corresponding to the different lag times according to the input variable and the output variable, where the mutual information corresponding to the different lag times is configured for determining a time dependency between the input variable and the output variable.

Further, a formula used for the step of obtaining the mutual information corresponding to the different lag times according to the input variable and the output variable includes:

$$MI ( x , y ) = \int \int dxdy μ ( x , y ) log \frac{μ ( x , y )}{{u}_{x} ( x ) {μ}_{y} ( y )} , \qquad ( 1 )$$

where MI(x, y) represents the mutual information, x represents the input variable, y represents the output variable, μ(x, y) represents a joint probability density function of the input variable and the output variable, uₓ(x) represents a marginal probability density function of the input variable, and μ_{y}(y) represents a marginal probability density function of the output variable.

Further, the step of establishing a tidal reach salinity prediction model according to the dry season data and the mutual information corresponding to the different lag times includes:
obtaining a model input variable and a model prediction variable according to the dry season data, where the model input variable includes tidal factor data, wind speed data, upstream runoff data, and the model prediction variable includes salinity data;
identifying time delay information that has impact on a change of salinity according to the mutual information corresponding to the different lag times, and selecting a lag time to be provided for model training;
establishing a decomposition framework, where the decomposition framework is configured for obtaining an intrinsic mode function through empirical mode decomposition according to the model input variable and the model prediction variable; and
obtaining the tidal reach salinity prediction model according to the decomposition framework, the lag time to be provided for model training, the model input variable, and the model prediction variable.

Further, formulas used for the decomposition framework configured for obtaining the intrinsic mode function through empirical mode decomposition according to the model input variable and the model prediction variable include:

$$m ( t ) = \frac{1}{2} ( {e}_{upper} ( t ) + {e}_{lower} ( t ) ) , \qquad ( 2 )$$

$$\begin{matrix}h ( t ) = x ( t ) - m ( t ) , \\ and\end{matrix} \qquad ( 3 )$$

$${IMF}_{k + 1} ( t ) = h ( t ) = {x}_{k} ( t ) - {m}_{k} ( t ) , \qquad ( 4 )$$

where IMFₖ₊₁(t) represents the intrinsic mode function; x(t) represents a current model input variable or a current model prediction variable; eᵤₚₚₑᵣ(t) represents an upper envelope formed by fitting all maximum points of x(t) using a cubic spline interpolation function; e_{lower}(t) represents a lower envelope formed by fitting all minimum points of x(t) using a cubic spline interpolation function; m(t) represents a mean line of the upper envelope and the lower envelope of x(t); h(t) represents a data sequence detail component; xₖ(t) represents a model input variable or a model prediction variable of a k^{th} iteration; and mₖ(t) represents a mean line of an upper envelope and a lower envelope corresponding to xₖ(t).

Further, the step of establishing a decomposition framework includes:
establishing an X framework, where the X framework is configured for performing empirical mode decomposition on the model input variable to generate first intrinsic mode functions; merging the first intrinsic mode functions into a new input dataset; training the input dataset and the model prediction variable using a random forest model to obtain a prediction result of the X framework;
establishing a Y framework; where the Y framework is configured for performing empirical mode decomposition on the model prediction variable to generate second intrinsic mode functions; setting a random forest model for each of the second intrinsic mode functions; performing training using the model input variable as an input of each random forest model; integrating a training result of each random forest model using each of direct addition, multiple linear regression and an artificial neural network, to obtain a prediction result of the Y framework;
establishing an XY framework, where the XY framework is configured for performing empirical mode decomposition on the model input variable and the model prediction variable respectively to generate third intrinsic mode functions; establishing a random forest model for each of the third intrinsic mode functions; performing training using the model input variable having been subjected to the empirical mode decomposition as an input of each random forest model; integrating a training result of each random forest model using each of direct addition, multiple linear regression and an artificial neural network, to obtain a prediction result of the XY framework; and
determining the X framework, the Y framework, and the XY framework as the decomposition framework.

In accordance with another aspect of the present disclosure, an embodiment further provides a system for predicting salinity in tidal reaches based on random forest and empirical mode decomposition, including:
a first module configured for acquiring dry season data;
   a second module configured for acquiring mutual information corresponding to different lag times according to the dry season data;
   a third module configured for establishing a tidal reach salinity prediction model according to the dry season data and the mutual information corresponding to the different lag times; and
   a fourth module configured for obtaining tidal reach salinity prediction information according to the tidal reach salinity prediction model.

In accordance with another aspect of the present disclosure, an embodiment further provides an apparatus for predicting salinity in tidal reaches based on random forest and empirical mode decomposition, including a memory, a processor, and a computer program stored in the memory and executable by the processor, where the computer program, when executed by the processor, causes the processor to implement the method for predicting salinity in tidal reaches based on random forest and empirical mode decomposition.

In accordance with another aspect of the present disclosure, an embodiment further provides a computer-readable storage medium having computer-executable instructions stored therein, where the computer-executable instructions are configured for causing a computer to execute the method for predicting salinity in tidal reaches based on random forest and empirical mode decomposition.

The embodiments of the present disclosure at least include the following beneficial effects: The present disclosure provides a method and system for predicting salinity of tidal reaches based on random forest and empirical mode decomposition. The method includes: acquiring dry season data; acquiring mutual information corresponding to different lag times according to the dry season data; establishing a tidal reach salinity prediction model according to the dry season data and the mutual information corresponding to the different lag times; and obtaining tidal reach salinity prediction information according to the tidal reach salinity prediction model. The present disclosure can effectively deal with the issues of multiple frequency components and lag time of complex signals, and improve the stability and prediction accuracy of the model.

The more accurate real-time salinity prediction results obtained through the method of the present disclosure can be directly applied to urban water supply and estuarine regulation systems. For example, when the prediction model identifies that the salinity will exceed a safety threshold in the coming days, the system may issue an early warning, enabling water management authorities to take measures such as: dispatching upstream reservoirs to increase discharge in order to dilute the saltwater intrusion; temporarily closing some intake gates and activating backup water sources; and optimizing industrial and domestic water-use scheduling along the riverbanks to ensure water supply safety.

### BRIEF DESCRIPTION OF DRAWINGS

FIG. 1 is a flowchart of a method for predicting salinity in tidal reaches based on random forest and empirical mode decomposition according to an embodiment of the present disclosure;

FIG. 2 is a flowchart of an X framework according to an embodiment of the present disclosure;

FIG. 3 is a flowchart of a Y framework according to an embodiment of the present disclosure;

FIG. 4 is a flowchart of an XY framework according to an embodiment of the present disclosure; and

FIG. 5 is a schematic diagram of a research area and data source stations according to an embodiment of the present disclosure.

### DETAILED DESCRIPTION

To make the objectives, technical solutions and advantages of the present disclosure clear, the present disclosure is further described in detail below with reference to the accompanying drawings and embodiments. It should be understood that the specific embodiments described herein are merely intended to explain the present disclosure and are not intended to limit the present disclosure. In the following description, when reference is made to the accompanying drawings, unless otherwise indicated, the same numerals in different drawings represent the same or similar elements. The illustrative implementations described in the following exemplary embodiments do not represent all implementations consistent with the embodiments of the present disclosure. Instead, they are merely examples of devices and methods consistent with certain aspects of the embodiments of the present disclosure as detailed in the appended claims.

It is understandable that the terms “first,” “second” and the like used in the present disclosure may be employed herein to describe various concepts. However, unless otherwise specified, these concepts are not limited by such terms. These terms are merely used to distinguish one concept from another. For example, without departing from the scope of the embodiments of the present disclosure, the first information may also be referred to as the second information, and similarly, the second information may also be referred to as the first information. Depending on the context, the words “if” and “in case” as used herein can be interpreted as “when,” “at the time of” or “in response to determining”.

As for the terms “at least one,” “a plurality of,” “each” and “any one” used in the present disclosure, “at least one” includes one, two or more, “a plurality of” includes two or more, “each” refers to every one of the corresponding plurality, and “any one” refers to any single one among the plurality.

Unless otherwise defined, meanings of all technical and scientific terms used in this specification are the same as those usually understood by those having ordinary skills in the art to which the present disclosure belongs. Terms used in this specification are merely intended to describe objectives of the embodiments of the present disclosure, but are not intended to limit the present disclosure.

Before the embodiments of the present disclosure are described in detail, a description is made on some nouns and terms in the embodiments of the present disclosure, and the terms in the embodiments of the present disclosure are applicable to the following definitions.
1) Mutual Information (MI), which measures the degree of interdependence between two random variables.
2) Empirical Mode Decomposition (EMD), which is an adaptive signal processing method.
3) Intrinsic Mode Function (IMF), which is a component obtained by EMD.
4) Random Forest (RF), which is an ensemble learning method.

The embodiments of the present disclosure will be further described in detail below with reference to the accompanying drawings.

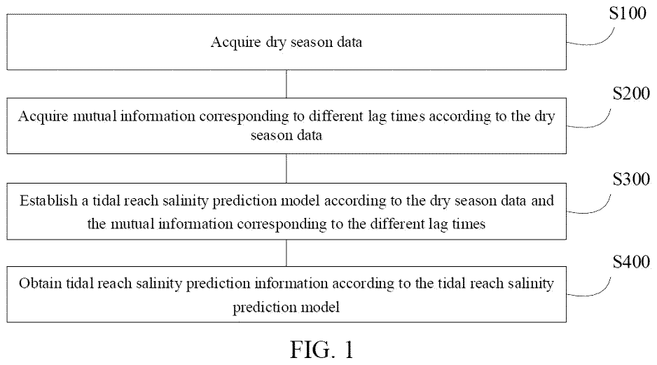

In accordance with one aspect of the present disclosure, an embodiment provides a method for predicting salinity in tidal reaches based on random forest and empirical mode decomposition. Referring to FIG. 1, the method for predicting salinity in tidal reaches based on random forest and empirical mode decomposition includes the following steps S100 to S400.

At S100, dry season data is acquired.

At S200, mutual information corresponding to different lag times is acquired according to the dry season data.

At S300, a tidal reach salinity prediction model is established according to the dry season data and the mutual information corresponding to the different lag times.

At S400, tidal reach salinity prediction information is obtained according to the tidal reach salinity prediction model.

At step S100, the dry-season data may be obtained by dedicated devices or instruments at the monitoring stations, for example but not limited to: 1) float-type tide gauges; 2) photoelectric anemometers; 3) vessel-mounted Acoustic Doppler Current Profilers (ADCPs) or sonar systems; 4) Conductivity-Temperature-Depth (CTD) instruments.

In an embodiment of the present disclosure, the step S100 of acquiring dry season data includes the following steps S110 to S120.

At S110, historical data of a target station is collected, where the historical data includes historical tidal level factor data, historical wind speed data, historical upstream runoff data, and historical salinity data,
the historical tidal level factor data includes a daily average tidal level, a daily maximum tidal level, a daily minimum tidal level, and a tidal range, and
the historical wind speed data includes an average wind speed, a maximum wind speed, and an extreme wind speed; and

At S120, the historical data is sorted into a daily scale and is sifted to obtain the dry season data, where the dry season data includes dry-season tidal level factor data, dry-season wind speed data, dry-season upstream runoff data, and dry-season salinity data. In an embodiment of the present disclosure, the step S200 of acquiring mutual information corresponding to different lag times according to the dry season data includes the following steps S210 to S220.

At S210, an input variable and an output variable are obtained according to the dry season data, where the input variable includes tidal level, wind speed, runoff, and the output variable includes salinity.

At S220, the mutual information corresponding to the different lag times is obtained according to the input variable and the output variable, where the mutual information corresponding to the different lag times is used for determining a time dependency between the input variable and the output variable.

As an optional implementation, mutual information is a metric for quantifying a dependency between two random variables. It measures the degree of information sharing among variables by calculating a difference between a joint probability distribution and a marginal probability distribution. In this embodiment, mutual information (MI) is used for determining lag times caused by different influencing factors (such as tidal level factor, wind speed, upstream runoff, etc.) to salinity prediction. The lag time refers to a time delay relationship between changes in influencing factors and changes in salinity.

In an embodiment of the present disclosure, a formula used for the step S220 of obtaining the mutual information corresponding to the different lag times according to the input variable and the output variable includes:

$$MI ( x , y ) = \int \int dxdy μ ( x , y ) log \frac{μ ( x , y )}{{u}_{x} ( x ) {μ}_{y} ( y )} , \qquad ( 1 )$$

where MI(x, y) represents the mutual information, x represents the input variable, y represents the output variable, μ(x, y) represents a joint probability density function of the input variable and the output variable, uₓ(x) represents a marginal probability density function of the input variable, and μ_{y}(y) represents a marginal probability density function of the output variable.

In an embodiment of the present disclosure, the step S300 of establishing a tidal reach salinity prediction model according to the dry season data and the mutual information corresponding to the different lag times includes the following steps S310 to S340.

At S310, a model input variable and a model prediction variable are obtained according to the dry season data, where the model input variable includes tidal factor data, wind speed data, upstream runoff data, and the model prediction variable includes salinity data.

At S320, time delay information that has impact on a change of salinity is identified according to the mutual information corresponding to the different lag times, and a lag time to be provided for model training is selected.

At S330, a decomposition framework is established, where the decomposition framework is configured for obtaining an intrinsic mode function through empirical mode decomposition according to the model input variable and the model prediction variable.

At S340, the tidal reach salinity prediction model is obtained according to the decomposition framework, the lag time to be provided for model training, the model input variable, and the model prediction variable.

As an optional implementation, the empirical mode decomposition (EMD) of the present disclosure decomposes an input signal (e.g., tidal level factor, wind speed, runoff, etc.) or an output signal (salinity data) into a series of intrinsic mode functions (IMFs). These IMFs represent different frequency components in the signal, and the decomposition process reveals the potential variation patterns of high-frequency and low-frequency components in data, making the data intuitive at multiple scales.

In an embodiment of the present disclosure, under different decomposition frameworks (such as X framework, Y framework, XY framework), the function of EMD is to decompose input data or output data (salinity data) into multiple IMFs and capture different hierarchical features in the signal. In the X framework, only the input variable is decomposed to generate IMFs, and the generated IMFs are inputted to a random forest model along with raw salinity data for training. In the Y framework, salinity data is decomposed into IMFs, which are synthesized using different prediction methods. The XY framework decomposes the input data and the output data respectively, ensuring that a more complex relationship between an input and an output can be captured.

As the core component of EMD, IMF is an important input for model training. Noise in the data can be effectively removed through IMFs, so that the random forest model can be trained using more intuitive and stationary input data, thereby improving the prediction accuracy of the model.

In an embodiment of the present disclosure, formulas used for the step S330 that the decomposition framework is configured for obtaining the intrinsic mode function through empirical mode decomposition according to the model input variable and the model prediction variable include:

$$m ( t ) = \frac{1}{2} ( {e}_{upper} ( t ) + {e}_{lower} ( t ) ) , \qquad ( 2 )$$

$$\begin{matrix}h ( t ) = x ( t ) - m ( t ) , \\ and\end{matrix} \qquad ( 3 )$$

$${IMF}_{k + 1} ( t ) = h ( t ) = {x}_{k} ( t ) - {m}_{k} ( t ) , \qquad ( 4 )$$

wherein IMFₖ₊₁(t) represents the intrinsic mode function; x(t) represents a current model input variable or a current model prediction variable; eᵤₚₚₑᵣ(t) represents an upper envelope formed by fitting all maximum points of x(t) using a cubic spline interpolation function; e_{lower}(t) represents a lower envelope formed by fitting all minimum points of x(t) using a cubic spline interpolation function; m(t) represents a mean line of the upper envelope and the lower envelope of x(t); h(t) represents a data sequence detail component; xₖ(t) represents a model input variable or a model prediction variable of a k^{th} iteration; and mₖ(t) represents a mean line of an upper envelope and a lower envelope corresponding to xₖ(t).

In an embodiment of the present disclosure, the step S330 of establishing a decomposition framework includes the following steps S331 to S334.

At S331, an X framework is established, where the X framework is configured for performing empirical mode decomposition on the model input variable to generate first intrinsic mode functions; merging the first intrinsic mode functions into a new input dataset; training the input dataset and the model prediction variable using a random forest model, to obtain a prediction result of the X framework.

At S332, a Y framework is established, where the Y framework is configured for performing empirical mode decomposition on the model prediction variable to generate second intrinsic mode functions; setting a random forest model for each of the second intrinsic mode functions respectively; performing training using the model input variable as an input of each random forest model; integrating a training result of each random forest model using each of direct addition, multiple linear regression, and an artificial neural network, to obtain a prediction result of the Y framework.

At S333, an XY framework is established, where the XY framework is configured for performing empirical mode decomposition on the model input variable and the model prediction variable respectively to generate third intrinsic mode functions; establishing a random forest model for each of the third intrinsic mode functions respectively; performing training using the model input variable having been subjected to the empirical mode decomposition as an input of each random forest model; integrating a training result of each random forest model using each of direct addition, multiple linear regression and an artificial neural network, to obtain a prediction result of the XY framework.

At S334, the X framework, the Y framework, and the XY framework are determined as the decomposition framework.

An objective of the present disclosure is to solve the defects in the conventional technology by providing a method for improved real-time prediction of salinity in estuarine tidal reaches based on a combination of empirical mode decomposition and random forest. By applying empirical mode decomposition to the preprocessing of non-stationary time series, extracting the intrinsic mode functions in the time series, and performing modeling using a random forest algorithm, the accuracy and stability of estuary salinity prediction are improved, thus enabling timely water management countermeasures to counter effects caused by the salinity.

As an optional implementation, an embodiment of the present disclosure provides a method for real-time prediction of salinity in estuarine tidal reaches based on random forest and empirical mode decomposition, which includes the following steps 1 to 4.

At step 1, data collection and variable selection are performed. Historical data of target stations is collected. The historical data mainly includes four types of data: tidal level factor data (T), including a daily average tidal level, a daily maximum tidal level, a daily minimum tidal level, and a tidal range; wind speed data (W), including an average wind speed, a maximum wind speed, and an extreme wind speed; upstream runoff data (R) and salinity data(S). The historical data is sorted into a daily scale and is sifted to obtain the dry season data (e.g., data from October 1st of this year to March 31st of the following year).

At step 2, lag times caused by influencing factors to salinity are determined. A position with a largest amount of information is found through calculating the Mutual information (MI) corresponding to different lag times, and then the lag time corresponding to the position is derived, and the time series is transformed according to the lag time. MI of two continuous random variables x and y is expressed by the following equation (1):

$$MI ( x , y ) = \int \int dxdy μ ( x , y ) log \frac{μ ( x , y )}{{u}_{x} ( x ) {μ}_{y} ( y )} , \qquad ( 1 )$$

where u(x, y) represents a joint probability density function of x and y; uₓ(x) and μ_{y}(y) respectively represent a marginal probability density function of x and a marginal probability density function of y. The MI is equal to zero if and only if the two variables are completely independent of each other, and a larger value of the MI indicates a stronger dependency between the variables. In an embodiment of the present disclosure, x includes tidal level, wind speed, and runoff, and y includes salinity.

At step 3, data preprocessing and model establishment are performed. Empirical mode decomposition (EMD) is performed on a model input variable (including tidal factor data, wind speed data, and upstream runoff data) and a model prediction variable (salinity data) to decompose the complex raw signals into a finite number of intrinsic mode functions (IMFs). There are two assumptions for IMFs: in the whole data segment, the number of extremum points and the number of zero crossing points must be equal or differ by no more than one; and at any time, an average value of an upper envelope formed by local maximum points and an lower envelope formed by local minimum points is zero, i.e., the upper and lower envelopes are locally symmetric with respect to the time axis.

The core of EMD is a sifting process for iteratively extracting IMFs. For example, for a current signal x(t), all maximum points of x(t) are found and fitted using a cubic spline interpolation function to form an upper envelope eᵤₚₚₑᵣ(t) of the raw data. Similarly, all minimum points of x(t) are found and fitted using a cubic spline interpolation function to form a lower envelope of the data. A mean line of the upper envelope and the lower envelope, i.e., an average envelope, (see formula (2)) is defined as m(t). The average envelope m(t) is subtracted from the raw data sequence to obtain a new data sequence detail component h(t). If the extracted detail component h(t) does not meet the definition of IMF, h(t) is determined as a new signal (see formula (3)). The above process of envelope construction and detail component extraction is repeated until h(t) meets the conditions of IMF, in which case h(t) is extracted as an IMF component. This process may be expressed by formula (4). The extracted IMF is removed from the raw signal to obtain a residual signal. The above steps are repeated for the residual signal until the residual signal becomes a monotonic function or a signal with very low frequency.

$$m ( t ) = \frac{1}{2} ( {e}_{upper} ( t ) + {e}_{lower} ( t ) ) \qquad ( 2 )$$

$$h ( t ) = x ( t ) - m ( t ) \qquad ( 3 )$$

$${IMF}_{k + 1} ( t ) = h ( t ) = {x}_{k} ( t ) - {m}_{k} ( t ) \qquad ( 4 )$$

xₖ(t) represents a signal of a k^{th} iteration, and mₖ(t) represents a corresponding mean line.

As an optional implementation, in an embodiment of the present disclosure, when EMD is performed on the dry season data, the following three decomposition frameworks may be used.

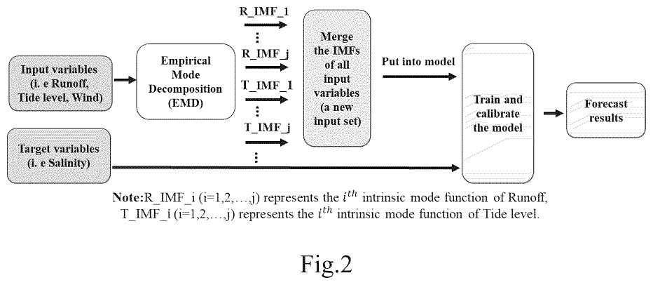

(1) Referring to FIG. 2, a process of the X framework is as follows. EMD is performed on only the model input variable to generate IMFs, and the generated IMFs (first intrinsic mode functions) are merged into a new input dataset. The model prediction variable is not processed, and a random forest model is established for training (where the model is called X-Only EMD).

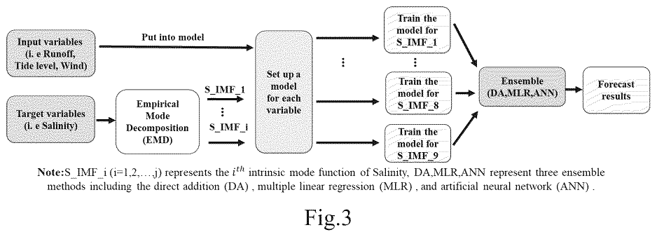

(2) Referring to FIG. 3, a process of the Y framework is as follows. Only the model prediction variable is decomposed. A random forest model is established for each of IMF (second intrinsic mode function) components obtained through decomposition. The model input variable is used as an input of each model without being processed. Prediction results of the models are respectively integrated using each of the following three methods: Direct Addition (DA), Multiple Linear Regression (MLR), and Artificial Neural Network (ANN). In the DA method, the prediction result is obtained by directly summing all predicted components (where the model is called Y-DA). In the MLR and ANN methods, the MLR and ANN models are trained to capture a relationship between the predicted components and original target variables in a training set, and the generated models are applied to generate predictions in a test set (where the models are respectively called Y-MLR and Y-ANN).

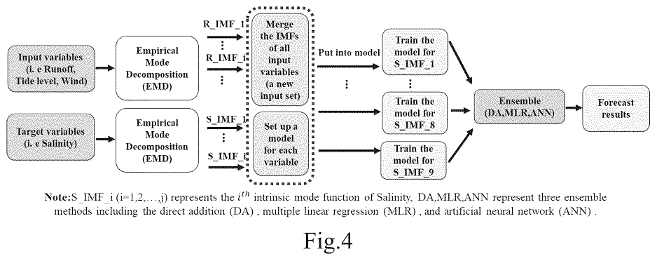

(3) Referring to FIG. 4, a process of the XY framework is as follows. The model input variable and the model prediction variable are decomposed respectively. For each of IMF (third intrinsic mode function) components obtained through decomposition, a random forest model is established for model training. The decomposed model prediction variable is used as an input of each model. Prediction results of the models are integrated respectively by three methods: DA, MLR, and ANN (where the models are correspondingly called XY-DA, XY-MLR, and XY-ANN).

To train and test the model, raw dry-season time series data is randomly divided into two parts, where 80% of the data is used as a training set and the remaining 20% is used as a test set. Parameters of the models are as shown in Table 1.

TABLE 1
Hyperparameter settings for RF and ANN models

| | Hyperparameter selection | | | |
|---|---|---|---|---|
| | n_{—} | max_{—} | min_samples_{—} | min_samples_{—} |
| Model | estimators | depth | split | leaf |
| RF | 200 | 15 | 2 | 1 |

| | hidden_layer_sizes | max_iter | random_state |
|---|---|---|---|
| ANN | 100 | 1000 | 42 |

At step 4, result evaluation is performed. The performance of the model is evaluated based on two evaluation metrics: coefficient of determination (R²) and root mean square error (RMSE). The R² statistic measures the proportion of variance in observation data explained by the model. The closer the value of R² is to 1, the better the model fitting effect (see formula (5)). The RMSE quantifies an average deviation between a predicted value and an observed value. A smaller value of the RMSE indicates higher prediction accuracy (see formula (6)).

$${R}^{2} = 1 - \frac{{\sum }_{i = 1}^{n} {( {y}_{i} - {\hat{y}}_{i} )}^{2}}{{\sum }_{i = 1}^{n} {( {y}_{i} - \overline{y} )}^{2}} \qquad ( 5 )$$

$$RMSE = \sqrt{\frac{1}{n} {{\sum }_{i = 1}}^{n} {( {y}_{i} - {\hat{y}}_{i} )}^{2}} \qquad ( 6 )$$

In the formulas, yᵢ represents the observed value, ŷᵢ represents the predicted value, and y̅ represents an average value of the observed values.

In a practical application case of the embodiments of the present disclosure, the Pearl River Delta of China is used as a research area.

At step 1, data collection and variable selection are performed.

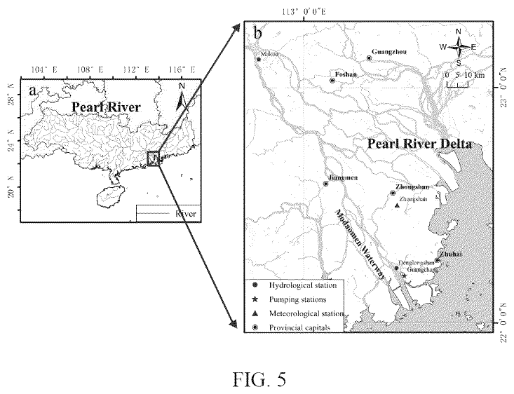

(1) Data collected in this case is daily-scale data of the dry season from 2006 to 2022 (October 1st of each year to March 1st of the following year), as shown in Table 2 below. The research area and data source stations are shown in FIG. 5. In FIG. 5, section a on the left is a map of the research area, and section b on the right is a schematic diagram of the data source stations.

The tidal level at the Denglongshan Hydrological Station is monitored using a float-type tide gauge. This traditional device measures tidal level based on the rise and fall of a float. The float is connected to a displacement sensor (such as an encoder or potentiometer) by a steel tape or pulley. When the water level changes, the vertical movement of the float is converted into an electrical signal, thereby recording variations of tidal level.

The wind speed at the Zhongshan Meteorological Station is monitored using a photoelectric anemometer. Utilizing photoelectric technology, a signal generator of the photoelectric anemometer includes a rotatable disk to affect the intensity of light transmission through rotation and a photoelectric transducer. When the wind cups rotate, the main shaft drives the disk to rotate, and the photoelectric transducer performs an optical scan to generate corresponding pulse signals. Within the wind-speed measurement range, the wind speed has a linear relationship with the pulse frequency, and the pulse frequency increases linearly with increasing wind speed.

The flow rate at the Makou Hydrological Station is monitored using a vessel-mounted Acoustic Doppler Current Profiler (ADCP) or a sonar system. After determining parameters through multiple sets of measured flow velocities, the flow rate of the entire cross-section of the Xijiang River can be calculated by measuring only half of the river width.

The salinity at the Guangchang Pumping Station is detected using a Conductivity-Temperature-Depth (CTD) instrument. By measuring the water's conductivity, temperature, and depth, the instrument automatically calculates the salinity, enabling automatic monitoring of water salinity.

TABLE 2
Research data

| | | | | Data |
|---|---|---|---|---|
| Category | Variable: | Abbreviation | Data source | range |
| Tidal level | Daily average | Tₘₑₐₙ | Denglongshan | Daily- |
| factor (T) | tidal level (m) | | hydrological | scale |
| | Daily maximum | Tₘₐₓ | station | data of |
| | tide level (m) | | | the dry |
| | Daily minimum | Tₘᵢₙ | | season |
| | tidal level (m) | | | from |
| | Tidal range (m) | T_{range} | | 2006 to |
| Wind | Average wind | Wₘₑₐₙ | Zhongshan | 2022 |
| speed (W) | speed (m/s) | | meteorological | |
| | Maximum wind | Wₘₐₓ | station | |
| | speed (m/s) | | | |
| | Extreme wind | Wₑₓₜᵣₑₘₑ | | |
| | speed (m/s) | | | |
| Upstream | Flow rate (m/s) | R | Makou | |
| runoff (R) | | | hydrological | |
| | | | station | |
| Salinity | Chlorine | S | Guangchang | |
| (S) | content (mg/L) | | pumping | |
| | | | station | |

At step 2, lag times caused by influencing factors to salinity are determined.

To determine an optimal lag time between the influencing factors and the chloride content (where the results are shown in Table 3), the following steps need to be performed.

(1) MI between a salinity time series corresponding to different lag times (typically 1 to 7 days) and influencing factors is calculated to evaluate a dependency between the variables in the case of each lag.

(2) An optimal lag time is determined by selecting the lag time with a highest MI value.

(3) The time series data is transformed by adjusting the lag times of the influencing factors, so as to be consistent with that corresponding to the determined optimal lag time.

TABLE 3
Lag effects of influencing factors in Guangchang pumping station

| | | Maximum | Lag |
|---|---|---|---|
| Variable: | Abbreviation | value of MI | time (d) |
| Daily average tidal level (m) | Tₘₑₐₙ | 0.0723 | 0 |
| Daily maximum tide level | Tₘₐₓ | 0.0527 | 3 |
| (m) | | | |
| Daily minimum tidal level | Tₘᵢₙ | 0.0832 | 1 |
| (m) | | | |
| Tidal range (m) | T_{range} | 0.0755 | 2 |
| Average wind speed (m/s) | Wₘₑₐₙ | 0.0339 | 2 |
| Maximum wind speed (m/s) | Wₘₐₓ | 0.0328 | 2 |
| Extreme wind speed (m/s) | Wₑₓₜᵣₑₘₑ | 0.0338 | 2 |
| Flow rate (m/s) | R | 0.41 | 1 |

At step 3, data preprocessing and model establishment are performed.

A PyEMD software package in a python environment is directly called to decompose the transformed model input variable and model prediction variable by EMD using each of the three frameworks previously discussed, and then models are respectively constructed for training.

At step 4, result evaluation is performed.

Compared with a conventional RF model, the XY-ANN model is the most robust method in estuary salinity prediction, followed by the XY-DA and XY-MLR models. By combining frequency components of two variable types, the XY framework enables the model to better capture implicit features and complex patterns of the model input variable and make more accurate predictions of low-frequency components (long-term trends) of the output variable. Such a dual decomposition method enhances the capability of the model to use structured and meaningful information extracted from the input signal and effectively copes with the fluctuation of the target output over time. The evaluation of the prediction result of the model is shown in Table 4.

TABLE 4
Evaluation indicators of the seven models under
three frameworks and the conventional RF model

| Model | R² | RMSE |
|---|---|---|
| X-Only-EMD | 0.85 | 642.48 |
| Y-DA | 0.64 | 1003.55 |
| Y-MLR | 0.63 | 1022.03 |
| Y-ANN | 0.62 | 1029.48 |
| XY-DA | 0.87 | 606.43 |
| XY-MLR | 0.88 | 574.58 |
| XY-ANN | 0.89 | 555.56 |
| RF | 0.64 | 1008.84 |

The key points of the embodiments of the present disclosure are as follows.

(1) Maximum mutual information (MI) for calculating the lag time: In the present disclosure, a maximum mutual information (MI) method is used to calculate the lag time between the input variable and the output variable. Compared with conventional methods, the MI method can better adapt to the time series of influencing factors of saltwater intrusion. Especially when dealing with nonlinear and complex time series dependencies, the MI method can more accurately identify the optimal lag time between variables, thereby significantly improving the accuracy and stability of prediction. Conventional methods may fail to consider the lag time or may select the lag only based on a simple linear relationship, resulting in limited prediction accuracy.

(2) Innovative application of the XY framework: In the present disclosure, more meaningful low-frequency and high-frequency components are extracted by using the XY framework, i.e., performing EMD on the input variable and the output variable respectively. Compared with frameworks that decompose only the input variable or the output variable, the XY framework more effectively captures the complex nonlinear relationship between input and output, thereby improving the prediction ability of the model.

(3) Hybrid model framework integrating EMD and RF: The present disclosure provides a method for estuary salinity prediction based on empirical mode decomposition (EMD) and random forest (RF). In the method, EMD is performed on an input variable and an output variable respectively to extract different frequency components in time series data, so that the defects of the existing technology in non-stationary data processing are solved. Then, an RF model is used for modeling, thereby improving the accuracy and stability of prediction.

The present disclosure adopts a hybrid model for estuary salinity prediction by combining a data preprocessing scheme with a machine learning technology, and therefore is advantageous over a single machine learning model. Conventional machine learning methods (such as support vector machine, artificial neural network, random forest, etc.) often have certain limitations in processing non-stationary data; while the present disclosure effectively deals with the issues of multiple frequency components and lag time of complex signals by calculating the lag time based on maximum mutual information (MI) and combining empirical mode decomposition (EMD) with random forest (RF) models, thereby improving the stability and prediction accuracy of the model.

The more accurate real-time salinity prediction results obtained through the method of the present disclosure can be directly applied to urban water supply and estuarine regulation systems. For example, when the prediction model identifies that the salinity will exceed a safety threshold in the coming days, the system may issue an early warning, enabling water management authorities to take measures such as: dispatching upstream reservoirs to increase discharge in order to dilute the saltwater intrusion; temporarily closing some intake gates and activating backup water sources; and optimizing industrial and domestic water-use scheduling along the riverbanks to ensure water supply safety.

In accordance with another aspect of the present disclosure, an embodiment further provides a system for predicting salinity in tidal reaches based on random forest and empirical mode decomposition, including:
a first module configured for acquiring dry season data;
a second module configured for acquiring mutual information corresponding to different lag times according to the dry season data;
a third module configured for establishing a tidal reach salinity prediction model according to the dry season data and the mutual information corresponding to the different lag times; and
a fourth module configured for obtaining tidal reach salinity prediction information according to the tidal reach salinity prediction model.

In accordance with another aspect of the present disclosure, an embodiment further provides an apparatus for predicting salinity in tidal reaches based on random forest and empirical mode decomposition, including a memory, a processor, and a computer program stored in the memory and executable by the processor, where the computer program, when executed by the processor, causes the processor to implement the method for predicting salinity in tidal reaches based on random forest and empirical mode decomposition.

The memory and the processor may be connected by a bus or in other ways. The memory, as a non-transitory computer-readable storage medium, may be configured for storing a non-transitory software program and a non-transitory computer-executable program. In addition, the memory may include a high-speed random access memory, and may also include a non-transitory memory, e.g., at least one magnetic disk storage device, flash memory device, or other non-transitory solid-state storage device. In some implementations, the memory may include memories located remotely from the processor, and the remote memories may be connected to the processor via a network. Examples of the network include, but are not limited to, the Internet, an intranet, a local area network, a mobile communication network, and combinations thereof.

In accordance with another aspect of the present disclosure, an embodiment further provides a computer-readable storage medium having computer-executable instructions stored therein, where the computer-executable instructions are configured for causing a computer to execute the method for predicting salinity in tidal reaches based on random forest and empirical mode decomposition.

Those having ordinary skills in the art can understand that all or some of the steps in the methods disclosed above can be implemented as software, firmware, hardware, and appropriate combinations thereof. Some or all physical components may be implemented as software executed by a processor, such as a central processing unit, a digital signal processor, or a microprocessor, or as hardware, or as an integrated circuit, such as an application-specific integrated circuit. Such software may be distributed on a computer-readable medium, which may include a computer storage medium (or non-transitory medium) and a communication medium (or transitory medium). As is known to those having ordinary skills in the art, the term “computer storage medium” includes volatile and nonvolatile, removable and non-removable media implemented in any method or technology for storage of information (such as computer-readable instructions, data structures, program modules, or other data). The computer storage medium includes, but not limited to, a Random Access Memory (RAM), a Read-Only Memory (ROM), an Electrically Erasable Programmable Read-Only Memory (EEPROM), a flash memory or other memory technology, a Compact Disc Read-Only Memory (CD-ROM), a Digital Versatile Disc (DVD) or other optical storage, a cassette, a magnetic tape, a magnetic disk storage or other magnetic storage device, or any other medium which can be used to store the desired information and which can be accessed by a computer. In addition, as is known to those having ordinary skills in the art, the communication medium typically includes computer-readable instructions, data structures, program modules, or other data in a modulated data signal such as a carrier or other transport mechanism, and can include any information passing medium.

Although some embodiments of the present disclosure are described above with reference to the accompanying drawings, these embodiments are not intended to limit the protection scope of the embodiments of the present disclosure. Any modifications, equivalent replacements and improvements made by those having ordinary skills in the art without departing from the scope and essence of the embodiments of the present disclosure shall fall within the protection scope of the embodiments of the present disclosure.

What is claimed is:

1. A method for predicting salinity in tidal reaches based on random forest and empirical mode decomposition, comprising:
acquiring dry season data;
acquiring mutual information corresponding to different lag times according to the dry season data;
establishing a tidal reach salinity prediction model according to the dry season data and the mutual information corresponding to the different lag times; and
obtaining tidal reach salinity prediction information according to the tidal reach salinity prediction model;
wherein the establishing the tidal reach salinity prediction model according to the dry season data and the mutual information corresponding to the different lag times comprises:
obtaining a model input variable and a model prediction variable according to the dry season data, wherein the model input variable comprises tidal factor data, wind speed data, upstream runoff data, and the model prediction variable comprises salinity data;
identifying time delay information that has impact on a change of salinity according to the mutual information corresponding to the different lag times, and selecting a lag time to be provided for model training;
establishing a decomposition framework, wherein the decomposition framework is configured for obtaining an intrinsic mode function through empirical mode decomposition according to the model input variable and the model prediction variable; and
obtaining the tidal reach salinity prediction model according to the decomposition framework, the lag time to be provided for model training, the model input variable, and the model prediction variable;
wherein the establishing the decomposition framework comprises:
establishing an X framework, wherein the X framework is configured for performing empirical mode decomposition on the model input variable to generate first intrinsic mode functions; merging the first intrinsic mode functions into a new input dataset; training the input dataset and the model prediction variable using a random forest model to obtain a prediction result of the X framework;
establishing a Y framework; wherein the Y framework is configured for performing empirical mode decomposition on the model prediction variable to generate second intrinsic mode functions; setting a random forest model for each of the second intrinsic mode functions; performing training using the model input variable as an input of each random forest model; integrating a training result of each random forest model using each of direct addition, multiple linear regression and an artificial neural network, to obtain a prediction result of the Y framework;
establishing an XY framework, wherein the XY framework is configured for performing empirical mode decomposition on the model input variable and the model prediction variable respectively to generate third intrinsic mode functions; establishing a random forest model for each of the third intrinsic mode functions; performing training using the model input variable having been subjected to the empirical mode decomposition as an input of each random forest model; integrating a training result of each random forest model using each of direct addition, multiple linear regression and an artificial neural network, to obtain a prediction result of the XY framework; and
determining the X framework, the Y framework, and the XY framework as the decomposition framework.

2. The method for predicting salinity in tidal reaches based on random forest and empirical mode decomposition of claim 1, wherein the acquiring the dry season data comprises:
collecting historical data of a target station, wherein the historical data comprises historical tidal level factor data, historical wind speed data, historical upstream runoff data, and historical salinity data;
the historical tidal level factor data comprises a daily average tidal level, a daily maximum tidal level, a daily minimum tidal level, and a tidal range;
the historical wind speed data comprises an average wind speed, a maximum wind speed, and an extreme wind speed; and
sorting the historical data into a daily scale and sifting the historical data to obtain the dry season data, wherein the dry season data comprises dry-season tidal level factor data, dry-season wind speed data, dry-season upstream runoff data, and dry-season salinity data.

3. The method for predicting salinity in tidal reaches based on random forest and empirical mode decomposition of claim 1, wherein the acquiring the mutual information corresponding to different lag times according to the dry season data comprises:
obtaining an input variable and an output variable according to the dry season data, wherein the input variable comprises tidal level, wind speed, runoff, and the output variable comprises salinity; and
obtaining the mutual information corresponding to the different lag times according to the input variable and the output variable, wherein the mutual information corresponding to the different lag times is configured for determining a time dependency between the input variable and the output variable.

4. The method for predicting salinity in tidal reaches based on random forest and empirical mode decomposition of claim 3, wherein a formula for obtaining the mutual information corresponding to the different lag times according to the input variable and the output variable comprises:
$$MI ( x , y ) = \int \int dxdy μ ( x , y ) log \frac{μ ( x , y )}{{u}_{x} ( x ) {μ}_{y} ( y )} , \qquad ( 1 )$$
wherein MI(x, y) represents the mutual information, x represents the input variable, y represents the output variable, μ(x, y) represents a joint probability density function of the input variable and the output variable, uₓ(x) represents a marginal probability density function of the input variable, and μ_{y}(y) represents a marginal probability density function of the output variable.

5. The method for predicting salinity in tidal reaches based on random forest and empirical mode decomposition of claim 1, wherein formulas for the decomposition framework configured for obtaining the intrinsic mode function through empirical mode decomposition according to the model input variable and the model prediction variable comprise:
$$m ( t ) = \frac{1}{2} ( {e}_{upper} ( t ) + {e}_{lower} ( t ) ) , \qquad ( 2 )$$
$$\begin{matrix}h ( t ) = x ( t ) - m ( t ) , \\ and\end{matrix} \qquad ( 3 )$$
$${IMF}_{k + 1} ( t ) = h ( t ) = {x}_{k} ( t ) - {m}_{k} ( t ) , \qquad ( 4 )$$
wherein IMFₖ₊₁(t) represents the intrinsic mode function; x (t) represents a current model input variable or a current model prediction variable; eᵤₚₚₑᵣ(t) represents an upper envelope formed by fitting all maximum points of x (t) using a cubic spline interpolation function; e_{lower}(t) represents a lower envelope formed by fitting all minimum points of x(t) using a cubic spline interpolation function; m(t) represents a mean line of the upper envelope and the lower envelope of x(t); h(t) represents a data sequence detail component; xₖ(t) represents a model input variable or a model prediction variable of a k^{th} iteration; and mₖ(t) represents a mean line of an upper envelope and a lower envelope corresponding to xₖ(t).

6. An apparatus for predicting salinity in tidal reaches based on random forest and empirical mode decomposition, comprising a memory, a processor, and a computer program stored in the memory and executable by the processor, wherein the computer program, when executed by the processor, causes the processor to implement the method for predicting salinity in tidal reaches based on random forest and empirical mode decomposition of claim 1.

7. A non-transitory computer-readable storage medium, having computer-executable instructions stored therein, wherein the computer-executable instructions are configured for causing a computer to execute the method for predicting salinity in tidal reaches based on random forest and empirical mode decomposition of claim 1.
